(19) United States
(12) Patent Application Publication
(10) Pub. No.: US 2025/0257306 A1
(43) Pub. Date: Aug. 14, 2025

(54) CELL DETACHMENT APPARATUS AND CELL DETACHMENT METHOD

(71) Applicant: CANON KABUSHIKI KAISHA, Tokyo (JP)

(72) Inventors: TATSUO FURUTA, Tokyo (JP); TAKANORI MATSUDA, Tokyo (JP); TAKAAKI FURUI, Tokyo (JP); RYOICHI YOSHIDA, Kanagawa (JP)

(21) Appl. No.: 19/195,002

(22) Filed: Apr. 30, 2025

Related U.S. Application Data
(63) Continuation of application No. PCT/JP2023/038840, filed on Oct. 27, 2023.

(30) Foreign Application Priority Data
Nov. 1, 2022 (JP) 2022-175808

(51) Int. Cl. C12M 1/26; C12M 3/00

(52) U.S. Cl. CPC C12M 33/08; C12M 23/48

(57) ABSTRACT

A cell detachment apparatus that detaches cells placed on a culture surface of a culture container for cells by applying vibration to the cells includes a vibrator and a vibration plate, wherein the vibrator, the vibration plate, an acoustic transmission medium, and the culture container are arranged to be stacked on each other in this order, and the cell detachment apparatus is configured so that an area where the acoustic transmission medium comes into contact with the vibration plate does not overlap an area where the vibrator comes into contact with the vibration plate in a stacking direction of the vibrator, the vibration plate, an acoustic transmission medium, and the culture container.

### CROSS-REFERENCE TO RELATED APPLICATIONS

This application is a Continuation of International Patent Application No. PCT/JP2023/038840, filed Oct. 27, 2023, which claims the benefit of Japanese Patent Application No. 2022-175808, filed Nov. 1, 2022, both of which are hereby incorporated by reference herein in their entirety.

### BACKGROUND

### Field

The present disclosure relates to a cell detachment apparatus and a cell detachment method.

### Background Art

In the fields of cellular medicine and regenerative medicine, it is necessary to culture a large number of cells, and in particular, there is a need for an efficient and stable supply of adherent cells that constitute a large part of biological tissues.

In culturing adherent cells, for example, cells are cultured on a culture container such as a polystyrene dish, detached from the culture container, and subjected to collection and washing processes, so that desired cells are acquired. To further increase the number of cells, a subculture operation is performed in which a portion of the obtained cells is transferred to a new culture container and cultured. In this series of processes, a detachment process for detaching cells from a culture container is generally performed by a method of detaching the cells by generating a flow in a liquid in the culture container using a pipette or the like. However, an angle of the pipette and the flow generated in the detachment process are different depending on a person, which is a barrier to an efficient and stable supply of cells.

According to Patent Literature 1, a cell collection device is discussed that supplies a detachment solution that reduces cell adhesiveness of a cultured cell group to a culture container containing the cultured cell group and vibrates the culture container using a vibration device to detach the cultured cell group from an inner wall of the culture container.

### CITATION LIST

### Patent Literature

Patent Literature 1: Japanese Patent Application Laid-Open No. 2008-79554

Here, when the vibration device generates vibration, the vibration device itself generates heat due to an internal loss such as mechanical friction. The heat generated by the vibration device is transferred to the cells via the culture container. This can reduce a survival rate of the cells.

A cooling device may be provided in the cell collection device in order to prevent the heat from being transferred to the cells, but this is not desirable because a device configuration becomes complicated and the device becomes expensive.

### SUMMARY

The present disclosure is directed to providing a cell detachment apparatus that reduces heat transferred to cells when vibration is applied to the cells and that provides the cells with a high survival rate.

According to an aspect of the present disclosure, a cell detachment apparatus that detaches cells placed on a culture surface of a culture container for cells by applying vibration to the cells includes a vibrator and a vibration plate, wherein the vibrator, the vibration plate, an acoustic transmission medium, and the culture container are arranged to be stacked on each other in this order, and the cell detachment apparatus is configured so that an area where the acoustic transmission medium comes into contact with the vibration plate does not overlap an area where the vibrator comes into contact with the vibration plate in a stacking direction of the vibrator, the vibration plate, an acoustic transmission medium, and the culture container.

According to another aspect of the present disclosure, a method for detaching cells placed on a culture surface of a culture container for cells by applying vibration to the cells includes applying vibration to the cells using a cell detachment apparatus that includes a vibrator and a vibration plate, an acoustic transmission medium, and the culture container in a state where the vibrator, the vibration plate, the acoustic transmission medium, and the culture container are arranged to be stacked on each other in this order, and applying vibration to the cells in a state where an area where the acoustic transmission medium comes into contact with the vibration plate does not overlap an area where the vibrator comes into contact with the vibration plate in the stacking direction.

According to the present disclosure, a cell detachment apparatus can be provided that reduces heat transferred to cells when vibration is applied to the cells and that provides the cells with a high cell survival rate.

Further features of the present disclosure will become apparent from the following description of exemplary embodiments with reference to the attached drawings.

### BRIEF DESCRIPTION OF THE DRAWINGS

FIG. 1A is a perspective view of a cell detachment apparatus according to an exemplary embodiment of the present disclosure.

FIG. 1B is an exploded perspective view of the cell detachment apparatus according to the exemplary embodiment of the present disclosure.

FIG. 1C is a cross-sectional view of the cell detachment apparatus according to the exemplary embodiment of the present disclosure.

FIG. 2A is a schematic view of a ring type vibrator used in the cell detachment apparatus according to the exemplary embodiment of the present disclosure.

FIG. 2B is a schematic view of the ring type vibrator used in the cell detachment apparatus according to the exemplary embodiment of the present disclosure.

FIG. 3 is a schematic side view of a central cross section of the cell detachment apparatus according to the exemplary embodiment of the present disclosure as viewed from a side.

FIG. 4 is a schematic side view of a central cross section of the cell detachment apparatus according to the exemplary embodiment of the present disclosure as viewed from the side.

FIG. 5 is a schematic side view of a central cross section of the cell detachment apparatus according to the exemplary embodiment of the present disclosure as viewed from the side.

FIG. 6A is a cross-sectional view of the cell detachment apparatus according to the exemplary embodiment of the present disclosure.

FIG. 6B is an exploded perspective view of the cell detachment apparatus according to the exemplary embodiment of the present disclosure.

FIG. 7A is a perspective view of a vibration plate in a deformed state that occurs when the vibrator used in the cell detachment apparatus according to the exemplary embodiment of the present disclosure is driven.

FIG. 7B is a cross-sectional view of the vibration plate in the deformed state that occurs when the vibrator used in the cell detachment apparatus according to the exemplary embodiment of the present disclosure is driven.

FIG. 7C is a cross-sectional view of the vibration plate in the deformed state that occurs when the vibrator used in the cell detachment apparatus according to the exemplary embodiment of the present disclosure is driven.

FIG. 8 is a cross-sectional view of an acoustic transmission medium used in the cell detachment apparatus according to the exemplary embodiment of the present disclosure.

### DESCRIPTION OF THE EMBODIMENTS

The present disclosure is described in detail below with reference to suitable exemplary embodiments.

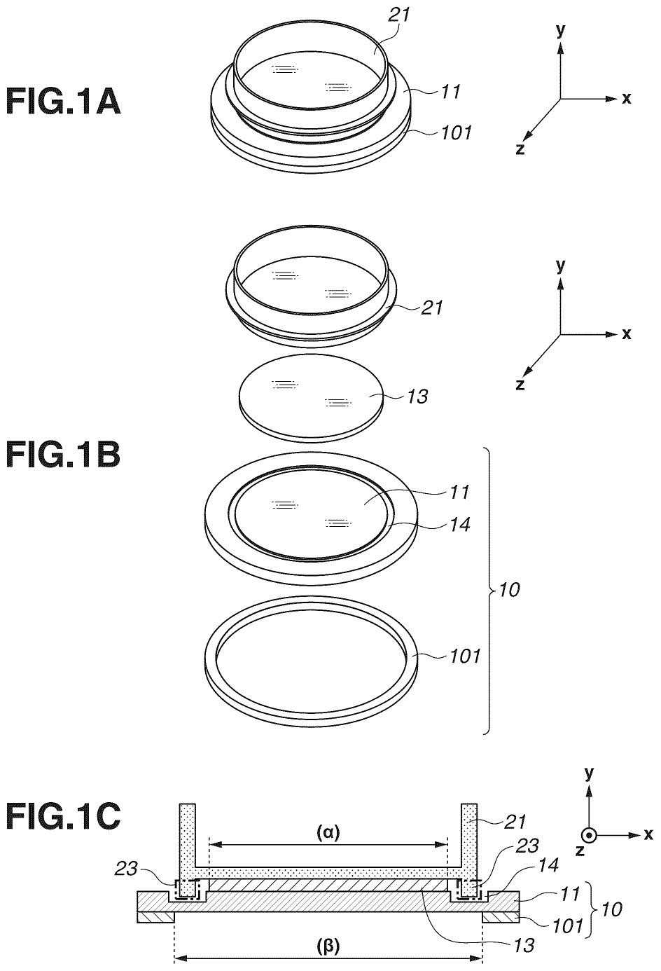

FIGS. 1A, 1B, and 1C are schematic views of a cell detachment apparatus according to an exemplary embodiment of the present disclosure. FIG. 1A is a schematic perspective view of a culture container 21 and the cell detachment apparatus as viewed from an oblique direction. FIG. 1B is an exploded perspective view of FIG. 1A and illustrates the culture container 21, an acoustic transmission medium 13, a vibration plate 11, and a vibrator 101 in a separated manner in a central axis direction of the ring type vibrator 101. FIG. 1C is a schematic side view of a central cross section of the cell detachment apparatus according to the exemplary embodiment of the present disclosure as viewed from a side.

As illustrated in FIGS. 1A to 1C, a cell detachment apparatus 10 according to the exemplary embodiment of the present disclosure includes the ring type vibrator 101 and the disk-shaped vibration plate 11. The cell detachment apparatus 10 according to the exemplary embodiment of the present disclosure drives the vibrator 101 in a state in which the vibrator 101, the vibration plate 11, the acoustic transmission medium 13, and the culture container 21 are arranged to be stacked on each other in this order. Vibration of the vibrator 101 is transmitted to the vibration plate 11, and vibration of the vibration plate 11 is further transmitted to the culture container 21 via the acoustic transmission medium 13, so that bending vibration is excited in the culture container 21. In this way, the cell detachment apparatus 10 applies the vibration to cells placed on a culture surface of the culture container 21 to detach the cells. Another member may be provided between any of the vibrator 101, the vibration plate 11, the acoustic transmission medium 13, and the culture container 21.

The vibration to be applied may be vibration in an ultrasonic frequency range. Here, the vibration in the ultrasonic frequency range is vibration with a frequency of 20 kHz or higher. The cell detachment apparatus 10 may have a configuration in which the acoustic transmission medium 13 is installed in advance or a configuration in which the acoustic transmission medium 13 is not included and a user installs the acoustic transmission medium 13 in the cell detachment apparatus 10. Further, the vibration plate 11 may have a marker (label, print, engraved mark, or the like) indicating a position where the acoustic transmission medium 13 is to be placed on the vibration plate 11. By the vibration plate 11 having the marker, the user can easily place the acoustic transmission medium 13 at an appropriate position.

Further, the cell detachment apparatus 10 may include a control unit that drives the vibrator 101 to apply vibration to the cells. The acoustic transmission medium 13 can also be referred to as an acoustic transmission material, an acoustic matching material, and a transmission medium.

Here, “detaching cells” refers to weakening adhesion of cells to a culture container so that the cells are in a state where they can be collected. At this time, the detached cells may exist as individual cells or may be in a state where a plurality of cells is adhered to each other. Further, as described below, the detached cells may be in a sheet form.

### (Ring Shape and Disk Shape)

In the present specification, “ring shape” refers to a form that has a disk shape with a predetermined thickness and a circular through hole and in which an outer circumference and the through hole are concentric. Further, in the present specification, “disk shape” refers to a circular shape with a predetermined thickness. In other words, a disk-shaped solid has a circular cross section. Outer peripheral shapes of the circular shape and the through hole are ideally perfect circles, but may also be ellipses, ovals, or the like as long as they can be schematically regarded as circles. In a case where the circular shape is not a perfect circle, its radius or diameter is defined by a length of the figure in a major axis direction, i.e., a major axis. In a case where the circular shape is not a perfect circle, its radius or diameter may also be determined by assuming a perfect circle having the same area.

Further, shapes with a part missing or a part protruding from the ring shape or disk shape are also included in the ring shape or disk shape in the present specification. Therefore, in a case where there is a slight deformation due to a manufacturing variation, the shape is included in the ring shape or disk shape in the present specification as long as the shape can be substantially regarded as a ring or a disk. In the case of a ring or a disk with a part missing or a part protruding, the inner and outer diameters of the ring and the radius and diameter of the disk are determined by assuming a perfect circle obtained by correcting a defective part and an abnormal part thereof.

The cell detachment apparatus according to the exemplary embodiment of the present disclosure that includes the ring type vibrator and the disk-shaped vibration plate is described, but the shape of each member of the cell detachment apparatus is not limited thereto. For example, both the vibrator and the vibration plate may have the disk shape, or the vibration plate may be a quadrangle and the vibrator may be a quadrangle with a central portion thereof removed.

### (Vibrator)

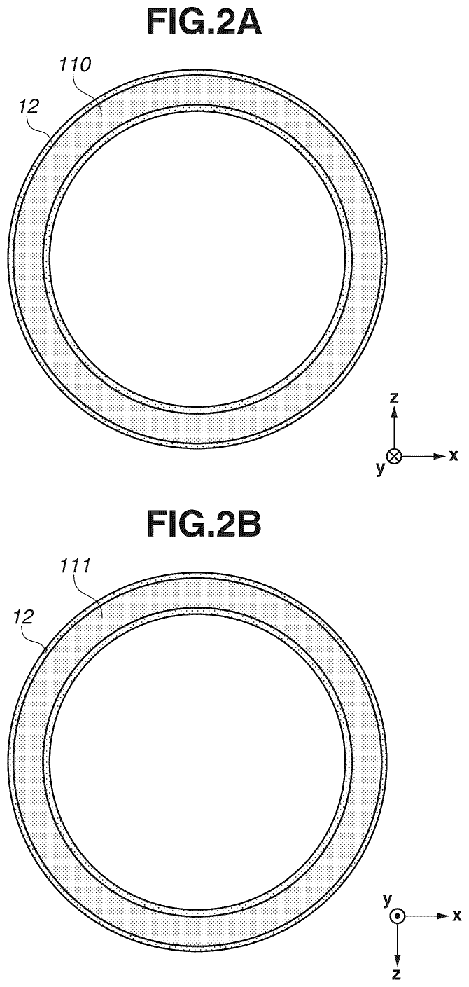

FIGS. 2A and 2B are schematic views of the ring type vibrator 101 used in the cell detachment apparatus 10 according to the exemplary embodiment of the present disclosure. FIG. 2A illustrates the ring type vibrator 101 as viewed from a direction opposite to a vibration plate side (a direction from the origin toward a y direction in FIG. 1C). FIG. 2B illustrates the ring type vibrator 101 as viewed from the vibration plate side (a direction from the origin toward a negative direction of the y axis in FIG. 1C).

The ring type vibrator 101 includes a ring-shaped piezoelectric material 12, a common electrode 111, and a driving electrode 110. The common electrode 111 is provided on a first surface (on the vibration plate side in the y direction in FIG. 1C) of the piezoelectric material 12. The driving electrode 110 is provided on a second surface, which is a surface opposite to the vibration plate side (in the negative direction of the y axis in FIG. 1C). When an alternating voltage is generated between the driving electrode 110 and the common electrode 111, the piezoelectric material 12 expands and contracts. Accordingly, a surface of the vibration plate 11 on a vibrator side expands and contracts in an in-plane direction, so that the vibration plate 11 causes bending vibration. In FIGS. 1A, 1B, and 1C, illustration of the common electrode 111 and the driving electrode 110 is omitted.

When the vibrator 101 is driven, an alternating electrode generated between the driving electrode 110 and the common electrode 111 may be continuously in an on state or may be switched to an off state at regular intervals to drive the vibrator 101 with a pause. By driving the vibrator 101 with a pause, it is possible to provide a time for the vibrator 101 to cool down and to further reduce the heat transferred to the cells. Further, when the vibrator 101 is driven, a driving frequency may be swept in a range including a natural frequency. When the driving frequency matches the natural frequency, a large amplitude is generated, and when it does not match, the amplitude is small, so that it is possible to provide a time to cool down the vibrator 101, and the heat transferred to the cells can be further reduced. Here, the natural frequency is defined as a frequency at which the vibration amplitude is maximum when the cell detachment apparatus is driven.

A type of the vibrator 101 may be a piezoelectric type or an electromagnetic type and is not particularly limited as long as it can generate vibration at a constant frequency. In a case of the piezoelectric type, the type can be selected from among a unimorph type, a bimorph type, and a bolt-clamped Langevin type. Further, a material and a structure of the vibrator 101 may be selected according to the frequency, force, and vibration amplitude to be generated.

### (Piezoelectric Material)

A form of the piezoelectric material 12 according to the exemplary embodiment of the present disclosure is not limited and may be any of ceramics, powder, single crystal, slurry, and the like, but is desirably ceramics. In the present specification, “ceramics” refers to an aggregate (also referred to as a bulk material) of crystal particles whose basic component is a metal oxide and that is sintered by heat treatment, namely, a polycrystal. Ceramics also include those processed after sintering.

Composition of the piezoelectric material 12 is not particularly limited. For example, the piezoelectric material 12 having at least one composition selected from the group consisting of, lead zirconate titanate, barium titanate, zirconate titanate, sodium bismuth titanate, potassium sodium niobate, sodium barium niobate titanate, and bismuth ferrate can be used for the vibrator 101. Further, the piezoelectric material 12 containing the above-described composition as a main component can be used for the vibrator 101 according to the exemplary embodiment of the present disclosure. It is desirable that a mechanical quality factor of the piezoelectric material 12 according to the exemplary embodiment of the present disclosure be 1000 or more.

### (Mechanical Quality Factor)

The mechanical quality factor is a coefficient that represents elastic loss due to vibration when the piezoelectric material 12 is evaluated as the vibrator 101, and magnitude of the mechanical quality factor is observed as sharpness of a resonance curve in impedance measurement. In other words, the mechanical quality factor is a constant that represents the sharpness of the resonance of the vibrator 101. When the mechanical quality factor of the piezoelectric material 12 is improved, an amount of heat generated in driving the vibrator 101 using the piezoelectric material 12 can be reduced.

### (Measurement of Piezoelectric Constant and Mechanical Quality Factor)

The mechanical quality factor of the piezoelectric material 12 can be obtained by calculation from a measurement result of resonance frequency and antiresonance frequency obtained using a commercially available impedance analyzer based on the Japan Electronics and Information Technology Industries Association standard (JEITA EM-4501). Hereinbelow, this method is referred to as a resonance-antiresonance method.

When the mechanical quality factor of the piezoelectric material 12 included in the vibrator 101 is 1000 or more, the loss during vibration is small, the amount of heat generated is small, and the heat is less likely to be transferred to the cells in the culture container 21.

### (Detection Electrode)

The vibrator 101 according to the exemplary embodiment of the present disclosure may include a detection electrode. The detection electrode has a function of monitoring a vibration state of the vibration plate 11 by detecting a positive piezoelectric effect caused by bending vibration occurring in the vibration plate 11 as a voltage change generated in a portion of the piezoelectric material 12 sandwiched between the common electrode 111 and the detection electrode. Since the vibrator 101 includes the detection electrode, it is possible to feed back the vibration state to a driving condition and to detect a failure.

### (Characteristic, Material, and Formation Method of Electrode)

The detection electrode, the common electrode 111, and the driving electrode 110 are made of a layered or film conductor having a resistance value of less than 10Ω, desirably less than 1Ω. The resistance value of the electrode can be evaluated by measurement with, for example, a multimeter (electrical tester). A thickness of each electrode is about 5 nm to 20 m. The material thereof is not particularly limited and may be any material that is normally used for the piezoelectric material 12.

Examples of materials for the electrodes include at least one metal selected from the group consisting of titanium (Ti), platinum (Pt), tantalum (Ta), iridium (Ir), strontium (Sr), indium (In), tin (Sn), gold (Au), aluminum (Al), iron (Fe), chromium (Cr), nickel (Ni), palladium (Pd), silver (Ag), and copper (Cu), and their compounds. The above-described electrodes may be made of one of these materials or may be made by laminating two or more of these materials. Among these materials, an Ag paste or Ag baked electrode, an Au/Ti sputtered electrode, and the like are desirable for the electrodes according to the exemplary embodiment of the present disclosure because of their small resistance values.

### (Shape of Vibration Plate)

The culture container 21 may have a protrusion portion 23 on a surface opposite to the surface on which the cells are provided that protrudes in a direction approximately perpendicular to the opposite surface. The protrusion portion 23 is provided, for example, at an edge portion of a bottom surface of the culture container 21. At this time, it is desirable that the vibration plate 11 be configured not to come into contact with the protrusion portion 23 when the culture container 21 is placed on the acoustic transmission medium 13. The protrusion portion 23 is a portion that protrudes toward an acoustic transmission medium side, such as a portion surrounded by an alternate long and short dash line in FIG. 1C. If the vibration plate 11 comes into contact with the protrusion portion 23 when the culture container 21 is placed on the acoustic transmission medium 13, heat is transferred from the vibration plate 11 without passing through the acoustic transmission medium 13, and thus the heat is easily transferred to the cells in the culture container 21.

As illustrated in FIG. 1B, it is desirable to provide a groove 14 in the vibration plate 11 so that the protrusion portion 23 does not come into contact with the vibration plate 11 since heat is not transferred from the vibration plate 11 to the culture container 21 without passing through the acoustic transmission medium 13. Specifically, as illustrated in FIG. 1C, when the acoustic transmission medium 13 and the culture container 21 are stacked in this order in the cell detachment apparatus, it is desirable that the groove 14 be provided in an area including a position where a position of the protrusion portion 23 is projected to the vibration plate 11 in a stacking direction of the acoustic transmission medium 13 and the culture container 21 (the y direction in FIG. 1C).

“The protrusion portion 23 of the culture container 21 does not come into contact with the vibration plate 11” substantially means that the protrusion portion 23 of the culture container 21 does not come into direct contact with a vibration surface of the vibration plate 11, and the protrusion portion 23 is desirably in midair. Further, there may be a cushioning material provided to an extent that prevents the vibration of the vibration plate 11 from being transmitted from the protrusion portion 23 to the culture container 21. Specifically, the cell detachment apparatus may be configured so that at least one of the acoustic transmission medium and the vibration plate comes into contact with the protrusion portion 23 via the cushioning material, and the acoustic transmission medium and the vibration plate are prevented from coming into contact with the protrusion portion 23 without passing through the cushioning material. It is desirable that the cushioning material be at least one of felt and sponge.

It is desirable that a surface of the vibration plate 11 on which the vibrator 101 is provided be a flat surface since vibration caused by expansion and contraction of the piezoelectric material 12 can be satisfactorily transmitted. A means for providing the vibrator 101 on the surface of the vibration plate 11 is not limited, but it is desirable to closely adhere the vibrator 101 thereto so as not to inhibit transmission of vibration or to adhere the vibrator 101 thereto via a highly elastic adhesive layer (not illustrated). For example, an epoxy-based resin is desirably used as the adhesive layer.

### (Material of Vibration Plate)

The vibration plate 11 is desirably an elastic member for a purpose of generating bending vibration together with the vibrator 101 and transmitting the vibration to the culture container 21 via the acoustic transmission medium 13. Further, from a viewpoint of elastic properties and workability, it is desirable that the vibration plate 11 include at least one of the group consisting of glass, metal, and quartz.

Examples of metals that can be used for the vibration plate 11 include aluminum, brass, Fe—Ni 36% alloy, and stainless steel (SUS).

### (Description of Standing Wave)

When an alternating voltage is applied to the driving electrode 110 of the vibrator 101, the piezoelectric material 12 of the vibrator 101 vibrates by expanding and contracting. Accordingly, the surface of the vibration plate 11 on the vibrator side expands and contracts in the in-plane direction, so that the vibration plate 11 causes bending vibration. An amplitude of the bending vibration forms a standing wave that reaches a maximum at a certain frequency of the alternating voltage. At this time, a portion where the amplitude becomes maximum is an antinode portion 17, and a portion where the amplitude becomes minimum is a node portion 18. While positions and the number of antinode portions 17 may be different depending on the frequency of the alternating voltage, it is desirable that the acoustic transmission medium 13 be provided to some or all of the antinode portions 17 of the standing wave generated in the vibration plate 11 together with the vibrator 101.

A surface of the acoustic transmission medium 13 that comes into contact with the vibration plate 11 is provided to some or all of the antinode portions 17 of the standing wave generated in the vibration plate 11 together with the vibrator 101, so that the vibration generated in the vibration plate 11 is efficiently transmitted to the culture container 21 via the acoustic transmission medium 13.

### (Method for Determining Position of Antinode of Vibrator)

In order to determine the position of the antinode portion 17 of the standing wave of any order that is generated when the vibrator 101 is driven, first, an input signal with a small voltage (for example, an alternating-current voltage of 1 to 10 V) is input to a drive phase electrode of the vibrator 101 in a state where only the vibrator 101 and the vibration plate 11 are present. Then, while a frequency of the input signal is changed using, for example, a laser Doppler vibrometer, the vibration amplitude is measured at a plurality of points on the surface of the vibrator 101 for each frequency, and accordingly the position can be determined. Specifically, the surface of the vibrator 101 is scanned at intervals of, for example, 1 mm, so that the vibration amplitude of the surface of the vibrator 101 can be measured. Among measurement points on the vibrator 101, a position where the vibration amplitude is minimum is regarded as the node portion 18, and a position where the vibration amplitude is maximum is regarded as the antinode portion 17.

### (Acoustic Transmission Medium)

As illustrated in FIG. 1C, the acoustic transmission medium 13 is provided on the surface of the vibration plate 11, and an area (α) where the acoustic transmission medium 13 comes into contact with the vibration plate 11 is located inside an inner circumference (β) of the vibrator 101. Here, “the area where the acoustic transmission medium 13 comes into contact with the vibration plate 11 is located inside the inner circumference of the vibrator 101” is not limited to a case where the area where the acoustic transmission medium 13 comes into contact with the vibration plate 11 is located in an area surrounded by the vibrator 101. “The area where the acoustic transmission medium 13 comes into contact with the vibration plate 11 is located inside the inner circumference of the vibrator 101” means that in a case where the area where the acoustic transmission medium 13 comes into contact with the vibration plate 11 is projected to a plane on which the vibrator 101 exists, a projected image is located inside the inner circumference of the vibrator 101. Since the acoustic transmission medium 13 is located inside the inner circumference of the vibrator 101, heat generated when the vibrator 101 is vibrated is less likely to be transferred to the acoustic transmission medium 13 via the vibration plate 11, and the heat is less likely to be transferred to the cells in the culture container 21.

When at least a part of the area where the acoustic transmission medium 13 comes into contact with the vibration plate 11 is located outside the inner circumference of the vibrator 101, the acoustic transmission medium 13 is located directly above the vibrator 101 with the vibration plate 11 in between. Thus, the heat generated by the vibrator 101 is transferred to the acoustic transmission medium 13 at a shortest distance via the vibration plate 11 and is easily transferred to the cells in the culture container 21.

The case where the vibrator 101 is ring-shaped is described, but the shape of the vibrator 101 is not limited to a ring. It is only necessary that the area where the acoustic transmission medium 13 comes into contact with the vibration plate 11 does not overlap with an area where the vibration plate 11 comes into contact with the vibrator 101. Here, it is assumed that the area where the acoustic transmission medium 13 comes into contact with the vibration plate 11 is projected to the plane on which the vibrator 101 exists. “The area where the acoustic transmission medium 13 comes into contact with the vibration plate 11 does not overlap with the area where the vibration plate 11 comes into contact with the vibrator 101” means that the area where the vibration plate 11 comes into contact with the vibrator 101 and the projected image of the area where the acoustic transmission medium 13 comes into contact with the vibration plate 11 do not overlap with each other. Since the area where the acoustic transmission medium 13 comes into contact with the vibration plate 11 does not overlap with the area where the vibration plate 11 comes into contact with the vibrator 101, the heat generated when the vibrator 101 is vibrated is less likely to be transferred to the acoustic transmission medium 13 via the vibration plate 11 and is less likely to be transferred to the cells in the culture container 21.

A means for providing the acoustic transmission medium 13 on the surface of the vibration plate 11 is not limited, but it is desirable to closely adhere the acoustic transmission medium 13 to the surface by applying pressure so as not to inhibit transmission of vibration or to adhere the acoustic transmission medium 13 to the surface via a highly elastic adhesive layer (not illustrated). In a case where the vibration plate 11 and the acoustic transmission medium 13 are pressure-bonded, the surface of the acoustic transmission medium 13 is smoothed to improve adhesion.

### (Installation Position of Acoustic Transmission Medium)

It is desirable that the area where the acoustic transmission medium 13 comes into contact with the vibration plate 11 be provided to some or all of the antinode portions 17 of the standing wave generated in the vibration plate 11 together with the vibrator 101. Since the acoustic transmission medium 13 comes into contact with the vibration plate 11 at the antinode portion 17, the vibration generated in the vibration plate 11 is efficiently transmitted to the culture container 21 via the acoustic transmission medium 13.

### (Shape of Acoustic Transmission Medium)

A shape of the acoustic transmission medium 13 is not particularly limited, but it is desirable that the shape be a disk shape from the viewpoint of efficiently transmitting the bending vibration of the vibration plate 11. Specifically, it is desirable that the acoustic transmission medium 13 have a circular cross section in a plane perpendicular to a direction in which the vibrator 101, the vibration plate 11, the acoustic transmission medium 13, and the culture container 21 are arranged to be stacked on each other. The standing wave of bending vibration generated in the disk-shaped vibration plate 11 by the ring type vibrator 101 has a large amplitude in a circular area centered on the antinode portion 17. Therefore, the acoustic transmission medium 13 formed in the disk shape can transmit the vibration in the area with the large amplitude to the culture container 21.

Here, a diameter of the disk-shaped acoustic transmission medium 13 is denoted by LN2, and a distance from a first antinode portion of the standing wave generated in the vibration plate 11 to a first node portion closest to the first antinode portion is denoted by LM. In order to more effectively transmit the vibration generated in the vibration plate 11, it is desirable that LN2/LM be less than 1.8, and more desirably 1.6 or less. Accordingly, since the vibration can be transmitted only from the area with a large amplitude, transmission efficiency of the vibration is good. Further, from the viewpoint of workability, the diameter of the acoustic transmission medium 13 is desirably 1 mm or more. Here, the diameter of the acoustic transmission medium 13 is a diameter of a cross section of the acoustic transmission medium 13 in a plane perpendicular to the direction in which the vibrator 101, the vibration plate 11, the acoustic transmission medium 13, and the culture container 21 are arranged to be stacked on each other.

The shape of the acoustic transmission medium 13 is not limited to a disk shape. A length of the acoustic transmission medium 13 in a direction perpendicular to the direction in which the vibrator 101, the vibration plate 11, the acoustic transmission medium 13, and the culture container 21 are arranged to be stacked on each other is defined as the length of the acoustic transmission medium 13. When the length of the acoustic transmission medium 13 is denoted by LN1, and the distance from the first antinode portion of the standing wave generated in the vibration plate 11 to the first node portion closest to the first antinode portion is denoted by LM, it is desirable that LN1/LM be less than 1.8, and more desirably 1.6 or less.

It is desirable that a thickness of a portion of the acoustic transmission medium 13 being in contact with the culture container 21 be 0.1 mm or more and 3 mm or less. Here, the thickness of the acoustic transmission medium 13 is the thickness of the acoustic transmission medium 13 in the direction in which the vibrator 101, the vibration plate 11, the acoustic transmission medium 13, and the culture container 21 are arranged to be stacked on each other. When the thickness is 3 mm or more, the vibration is attenuated within the acoustic transmission medium 13, and there is a risk that the vibration of the vibrator 101 is not transmitted to the culture container 21. Further, when the thickness of the acoustic transmission medium 13 is less than 0.1 mm, it is difficult to manufacture the acoustic transmission medium 13, so that the thickness is desirably 0.1 mm or more.

It is desirable that the surface of the acoustic transmission medium 13 on the side that comes into contact with the culture container 21 include a convex curved surface. While it is desirable that the bottom of the culture container 21 is a plane, a commercially available culture container 21 that is generally used for cell culture may have a bottom surface warped in a direction opposite to the acoustic transmission medium 13.

When the acoustic transmission medium 13 is flat, air may enter between the acoustic transmission medium 13 and the bottom surface of the culture container 21. When air remains, the vibration of the vibration plate 11 may be reflected at an interface between the acoustic transmission medium 13 and the air or at an interface between the culture container 21 and the air, or the vibration may be attenuated by the air, so that the vibration of the vibrator 101 is less likely to be transmitted to the culture container 21. Therefore, by the surface of the acoustic transmission medium 13 that comes into contact with the culture container 21 including the convex curved surface, it becomes difficult for air to enter between the culture container 21 and the acoustic transmission medium 13, and the vibration of the vibrator 101 is easily transmitted to the culture container 21.

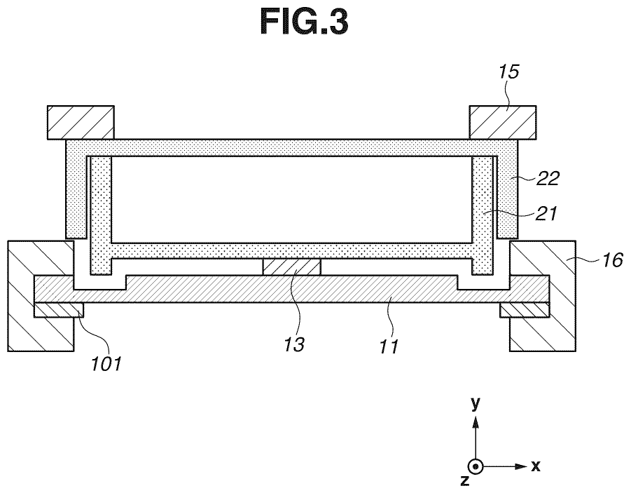

FIG. 3 is a schematic side view of a central cross section of the cell detachment apparatus 10 according to the exemplary embodiment of the present disclosure as viewed from a side. The culture container 21 placed on the cell detachment apparatus 10 is covered with a lid 22, and a ring-shaped weight 15 is placed on the lid 22. Further, a ring-shaped fixing member 16 fixes the vibration plate 11 and the vibrator 101 by sandwiching them. The weight 15 brings the culture container 21 and the acoustic transmission medium 13 into pressure contact with each other, so that it becomes difficult for air to enter between the culture container 21 and the acoustic transmission medium 13, and the vibration of the vibrator 101 is easily transmitted to the culture container 21.

### (Material of Acoustic Transmission Medium)

It is desirable that the acoustic transmission medium 13 be made of a material that easily transmits the vibration of the vibrator 101 to the culture container 21 and contains a polymer material that does not allow air to easily enter between the acoustic transmission medium 13 and the culture container 21 and has high tackiness. For example, at least one selected from the group consisting of rubber, gel, and viscoelastic material can be used as a material of the acoustic transmission medium 13. Among these, rubber is a desirable material because it has a high vibration transmission property and is inexpensive. Further, it is desirable that a thermal conductivity of the acoustic transmission medium 13 be 0.4 W/(m·K) or less. Since the thermal conductivity of the acoustic transmission medium 13 is 0.4 W/(m·K) or less, the heat generated by the vibrator 101 is less likely to be transferred to the culture container 21 even though it is transferred to the acoustic transmission medium 13 via the vibration plate 11, and accordingly, the heat is not transferred to the cells in the culture container 21. Further, silicone rubber has a physical property of less attenuation in the frequency band of the driving frequency, and thus has high transmission efficiency of vibration and can reduce heat generation of the acoustic transmission medium 13 itself. For these reasons, it is desirable that the material of the acoustic transmission medium 13 contain silicone rubber.

### (Maintaining Positional Relationship During Driving)

The above-described configuration can reduce the heat transfer from the vibrator 101. However, when an arrangement of three components, the vibrator 101, the vibration plate 11, and the acoustic transmission medium 13 in a circumferential direction changes during driving, the transmission efficiency of the vibration may decrease, or the heat from the vibrator 101 may be transferred more easily. Thus, in a case where the cell detachment apparatus 10 according to the exemplary embodiment of the present disclosure is driven, it is desirable that the three components, the ring type vibrator 101, the disk-shaped vibration plate 11, and the acoustic transmission medium 13 be fixed to each other in arrangement thereof in the circumferential direction. In order to fix them, the fixing member 16 illustrated in FIG. 3 may be used, or they may be bonded to each other using an adhesive. Further, they may each be fixed to another component with an adhesive or the like. The fixing member 16 is desirably made of felt, sponge, or rubber so as not to inhibit the vibration of the vibrator 101 and the vibration plate 11.

Further, a cushioning material such as felt and sponge, which is less likely to transmit the vibration, may be provided between the vibration plate 11 and the culture container 21. By providing the cushioning material, it is possible to prevent the culture container 21 from tilting and coming into direct contact with the vibration plate 11. Furthermore, in addition to the fixing member 16, the cushioning material such as felt and sponge, which is less likely to transmit vibration, may be provided below the vibration plate 11. By providing the cushioning material, it is possible to reduce the attenuation of vibration caused by the vibrator 101 or the vibration plate 11 coming into contact with a workbench.

### (Culture Container)

The culture container 21 to be placed on the cell detachment apparatus 10 is not particularly limited. Any container may be used as long as desired cells can be cultured. A material of the culture container 21 may be at least one selected from the group consisting of, for example, polyethylene, polypropylene, polycarbonate, polystyrene, polyvinyl chloride, nylon, polyurethane, polyurea, polylactic acid, polyglycolic acid, polyvinyl alcohol, polyvinyl acetate, poly(meth)acrylic acid, poly(meth)acrylic acid derivative, polyacrylonitrile, poly(meth)acrylamide, poly(meth)acrylamide derivative, polysulfone, cellulose, cellulose derivative, polysilicone, polymethylpentene, glass, and metal. Polystyrene is desirable among these materials.

The culture container 21 may be of types such as a dish, a flask, and a well plate. Particularly, dishes manufactured by Corning Inc., Thermo Fisher Scientific Inc., and AGC Techno Glass Co., Ltd. (Iwaki) of Japan are used as general-purpose products. A culture surface of the culture container is a surface surrounded by a side wall, and in a case where cells are cultured, the cells adhere to the culture surface.

### (Cell Culture Conditions)

Cell culture conditions can be appropriately selected according to cells to be cultured. In general, an appropriate culture medium is added to the culture container 21, and cells are seeded therein at about 1.0×10¹ to 5.0×10⁴ cells/cm² and cultured in an environment with a temperature of 37° C. and a CO₂ concentration of 5%. At this time, it is desirable to culture until a cell confluency in the culture container 21 reaches about 70 to 80%, i.e., what is called a sub-confluent state. Further, the cells may be cultured until reaching a confluent state in which the cells cover the culture surface in a sheet form (cell sheet).

### (Detachment Test)

The cell detachment apparatus 10 can be applied to various types of cells. The cells include, for example, at least one selected from the group consisting of a Chinese hamster ovary (CHO) cell, a mouse connective tissue L929 cell, a mouse skeletal muscle myoblast (C2C12 cell), a human fetal lung-derived normal diploid fibroblast (TIG-3 cell), a human fetal kidney-derived cell (HEK293 cell), a human alveolar basal epithelial adenocarcinoma-derived A549 cell, a human cervical cancer-derived HeLa cell, an epithelial cell, an endothelial cell, a skeletal muscle cell, a smooth muscle cell, a cardiac muscle cell, a neuron cell, a glial cell, a fibroblast cell, a liver parenchymal cell, a liver non-parenchymal cell, a fat cell, an induced pluripotent stem (iPS) cell, an embryonic stem (ES) cell, an embryonic germ (EG) cell, an embryonal carcinoma (EC) cell, a mesenchymal stem cell, a liver stem cell, a pancreatic stem cell, a skin stem cell, a muscle stem cell, a germ stem cell, a progenitor cell of each tissue, and cells differentiated and induced therefrom.

A culture medium for the cultured cells needs to be replaced with a detachment solution in a cell detachment process. The detachment solution usually contains a proteolytic enzyme. The proteolytic enzyme may be at least one selected from the group consisting of, for example, trypsin, accutase, collagenase, natural protease, chymotrypsin, elastase, papain, pronase, and recombinant thereof. However, there is a concern that the proteolytic enzyme may degrade a membrane protein included in the cells. Degradation of the membrane protein may reduce properties such as cell adhesiveness. In contrast, a cell detachment method according to the present disclosure does not use the proteolytic enzyme, and even when it does, only a very small amount of the proteolytic enzyme is used to achieve the same effect, so that it has an effect of reducing the degradation of a cell membrane protein. The detachment solution may contain a culture medium component in order to suppress a reduction of the cell membrane protein.

The culture container 21 in which the culture medium is replaced with the detachment solution is placed in the cell detachment apparatus 10, and the cell detachment apparatus 10 is driven in a state of being held at a constant temperature, for example, 37° C., in an incubator. Driving is performed for a driving time sufficient for cell detachment. After driving, detached cells are collected using a dropper, and cells that cannot be collected and are still attached to the culture container 21 are collected by a pipetting method. A detachment rate is a ratio of the number of cells detached by vibration to the total number of cells detached by both vibration and the pipetting method. A survival rate is a ratio of cells that are alive among the collected cells.

Further, residual cells remaining on the bottom surface of the culture container 21 after the cell detachment can be observed using scattered light by backlight.

### (Other Configurations)

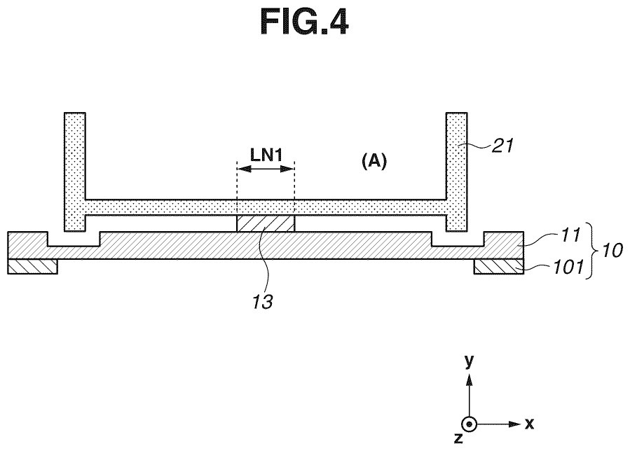

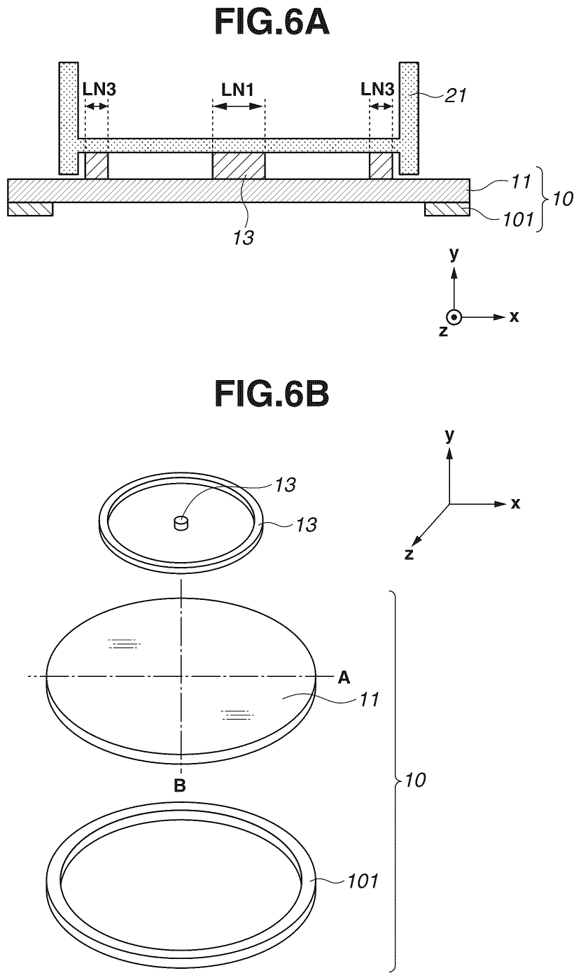

FIGS. 4, 5, 6A, and 6B are schematic drawings illustrating the cell detachment apparatus according to exemplary embodiments of the present disclosure. FIGS. 4, 5, and 6A are schematic side views of the central cross section of the cell detachment apparatus according to the exemplary embodiments of the present disclosure viewed from the side. FIG. 6B is an exploded perspective view of the cell detachment apparatus 10 in FIG. 6A and illustrates the acoustic transmission medium 13, the vibration plate 11, and the vibrator 101 in a separated manner in the central axis direction of the ring type vibrator 101.

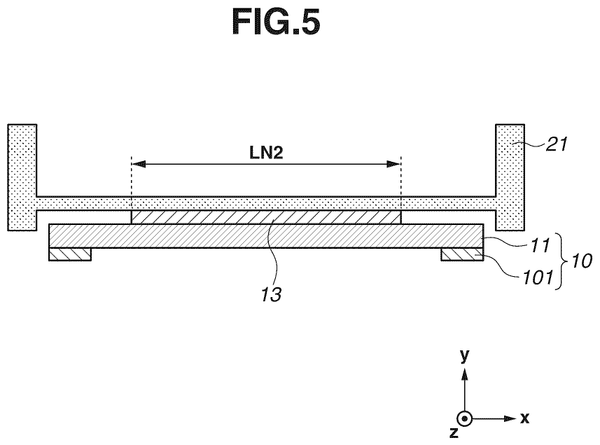

Each figure illustrates the exemplary embodiment in which the shapes of the acoustic transmission medium 13 and the vibration plate 11 are different. FIG. 4 illustrates the disk-shaped acoustic transmission medium 13 of which the diameter is smaller than that illustrated in FIGS. 1A to 1C. FIG. 5 illustrates the disk-shaped vibration plate 11 without a groove. Further, a plurality of acoustic transmission media may be installed as the acoustic transmission medium 13, and, for example, the disk-shaped acoustic transmission medium 13 and the ring-shaped acoustic transmission medium 13 may be installed. When a width of the ring-shaped acoustic transmission medium 13 is denoted by LN3, and the distance from the first antinode portion of the standing wave generated in the vibration plate 11 to the first node portion closest to the first antinode portion is denoted by LM, it is desirable that LN3/LM be less than 1.8, and more desirably 1.6 or less. Here, the width of the ring-shaped acoustic transmission medium 13 is the width of the ring-shaped acoustic transmission medium 13 in the direction perpendicular to the direction in which the vibrator 101, the vibration plate 11, the acoustic transmission medium 13, and the culture container 21 are arranged to be stacked on each other, i.e., a distance between an outer circumference and an inner circumference in a radial direction of the ring-shaped acoustic transmission medium 13.

FIGS. 6A and 6B illustrate an example in which two acoustic transmission media 13 are installed. The effect of the present disclosure can be acquired in any of the exemplary embodiments.

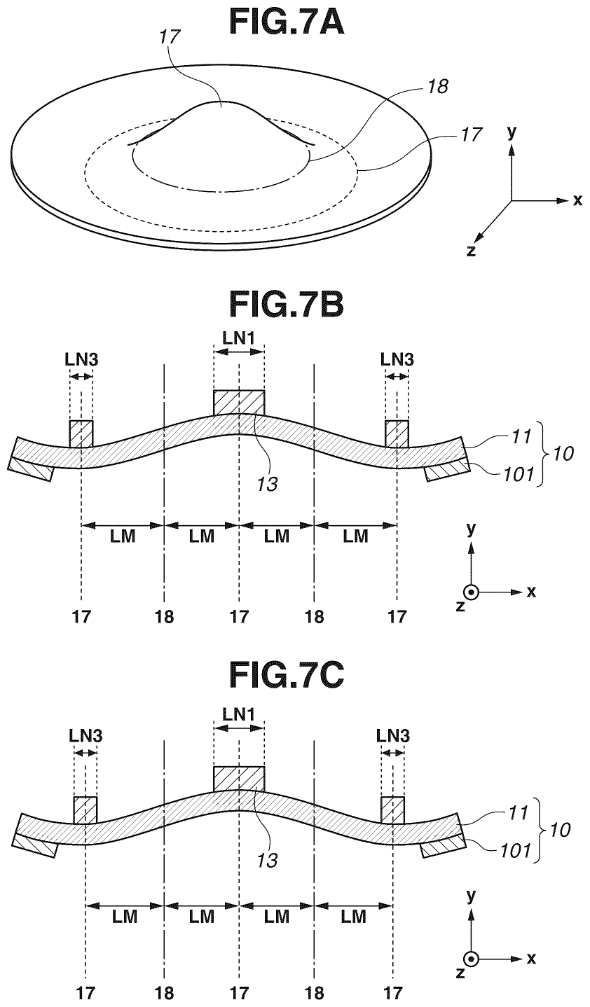

FIGS. 7A, 7B, and 7C illustrate deformed states when the amplitude of the third-order standing wave generated at a time of driving the vibrator 101 in FIGS. 6A and 6B is maximum. FIG. 7A is a perspective view of the vibration plate 11 alone. FIG. 7B is a schematic side view of the central cross section (cross section A in FIG. 6B) in the deformed state as viewed from the side. FIG. 7C is a schematic side view of the central cross section (cross section B in FIG. 6B) in the deformed state as viewed from the side.

However, a deformation amount is illustrated with an exaggerated magnification. A central portion and dashed line portions in FIG. 7A are the antinode portions 17. Node portions 18 are indicated by alternate long and short dash lines in FIGS. 7B and 7C.

As illustrated in FIGS. 7A, 7B, and 7C, the acoustic transmission media 13 are provided at positions corresponding to the antinode portions 17 of the standing wave on the vibration plate 11, so that the vibration of the vibrator 101 and the vibration plate 11 is efficiently transmitted to the culture container 21 via the acoustic transmission media 13.

### EXAMPLES

A first example according to the first exemplary embodiment is described below.

The cell detachment apparatus 10 in FIG. 4 was used in the first example. For the culture container 21, Φ60 mm dish manufactured by Corning Inc. was used. For the vibration plate 11 of the cell detachment apparatus 10, a disk-shaped glass having a diameter of 70 mm, a thickness of 4 mm, and the groove 14 formed therein so as not to contact the protrusion portion 23 of the culture container 21 was used. The dimensions of the groove 14 were an inner diameter of 50 mm, a width of 5 mm, and a depth of 1 mm. The material composition of the piezoelectric material 12 was piezoelectric zirconate titanate (PZT), an outer diameter thereof was 70 mm, which was the same as the vibration plate, an inner diameter thereof was 57 mm, and a thickness thereof was 2 mm. The driving electrode 110 and the common electrode 111 were made of silver. Next, the vibrator and the vibration plate were bonded with an epoxy adhesive, the vibrator was swept at a voltage of 50 V and a frequency of 22 to 27 kHz, and the vibration amplitude at each frequency was measured using a laser Doppler vibrometer (AT7200, manufactured by Graphtec Corporation). As a result, it was confirmed that the standing wave illustrated in FIG. 7A was generated at 25.6 kHz.

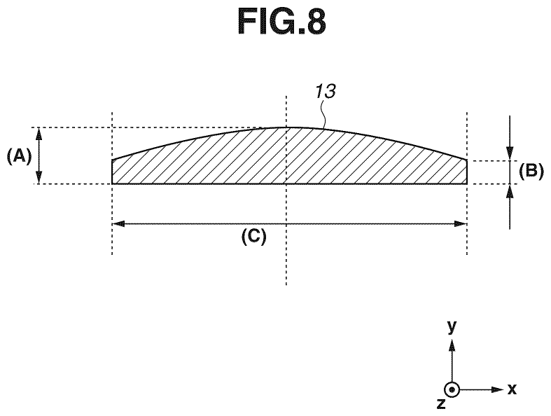

Next, the disk-shaped acoustic transmission medium 13 illustrated in FIG. 8 was placed on the central portion of the vibration plate 11. FIG. 8 is a side view of a central cross section of the disk-shaped acoustic transmission medium 13 in which a surface in contact with the culture container 21 has a convex curved surface. Dimensions are: central height (A) 0.9 mm, side height (B) 0.4 mm, and diameter (C) 48 mm, and a material thereof is silicone rubber with a hardness of 30°. The cell detachment apparatus 10 was assembled as illustrated in FIG. 3. Φ60 mm dish from Corning Inc. for the culture container 21 in which cells were cultured was placed on the silicone rubber placed on the vibration plate 11, and a 300 g weight 15 was placed thereon. At this time, it was visually confirmed that there was no air between the acoustic transmission medium 13 and the culture container 21 and that they were in close contact with each other over their entire surfaces.

For cell culture, Chinese hamster ovary (CHO) cells were seeded in a (D 60 mm dish from Corning Inc. at a density of 10,000 cells/cm² and cultured in an environment with a temperature of 37° C. and a CO₂ concentration of 5%. A culture medium used was Ham's F12 (manufactured by Thermo Fisher Scientific Inc.) supplemented with 10% Fetal Bovine Serum (manufactured by Sigma-Aldrich Corporation) and 1% penicillin-streptomycin (10,000 U/ml, manufactured by Thermo Fisher Scientific Inc.). The cell culture was performed for 48 hours, and a state of the cells was observed using a phase-contrast microscope to confirm cell adhesion and proliferation. A cell confluency in the dish was about 80%.

The culture medium in the dish was removed, and the dish was washed with phosphate buffered saline (PBS(−), manufactured by Thermo Fisher Scientific Inc.). Then, phosphate buffered saline (PBS(−), manufactured by Thermo Fisher Scientific Inc.) was added to the dish as a detachment solution, and the cells were immersed in the detachment solution for 3 minutes. Then, the culture container 21 was placed in the cell detachment apparatus 10, and the cells were detached using frequency sweep vibration (frequency 22-27 kHz, frequency sweep period 1 second, voltage 100 V) for 1 minute and 30 seconds.

At this time, the temperature of the detachment solution was measured with a thermocouple, and a temperature increase from before detachment was 1° C. or less.

After the detached cells were collected, the number of cells was measured using a hemocytometer, and the survival rate was calculated using a viability assay using trypan blue staining. First, a suspension of detached cells was mixed with 0.4% trypan blue solution at a ratio of 1:1 to stain dead cells contained in the suspension. Subsequently, the suspension was injected into a hemocytometer (manufactured by NanoEntek, DHC-N01), and the numbers of living cells and dead cells on a grid of the hemocytometer was counted by microscopic observation. The ratio of the number of living cells counted to the total number of cells was calculated as the survival rate.

Further, after the ultrasonic detachment, all cells that could not be detached by ultrasound were detached from the dish using a cell scraper. A suspension of detached cells was injected into the hemocytometer (manufactured by NanoEntek, DHC-N01), and the number of detached cells was measured by microscopic observation. The total number of cells detached by ultrasound and the number of cells subsequently detached with the cell scraper were added to obtain the total number of detached cells, and the ratio of the number of cells detached by ultrasound to the total number of detached cells was defined as a detachment rate and calculated. As a result, the survival rate was 98.0% and the detachment rate was 95.7%. The survival rate and the detachment rate of 90% or more were determined to be good.

Exemplary embodiments of the present disclosure may also be implemented by a computer of a system or apparatus (e.g., an application specific integrated circuit (ASIC)) that reads and executes computer-executable instructions (e.g., one or more programs) recorded on a storage medium performing one or more of the exemplary embodiments, and/or by a computer of a system or apparatus including one or more circuits that perform one or more functions of the exemplary embodiment, and for example, by reading and executing computer-executable instructions from a storage medium to perform the one or more functions of the exemplary embodiments, and/or by a method performed by a computer of a system or apparatus by controlling one or more circuits to perform the one or more functions of the exemplary embodiments. The computer may include one or more processors (e.g., central processing unit (CPU), micro processing unit (MPU)) and may include a network of a separate computer or a separate processor to read and execute the computer executable instructions. The computer executable instructions may be provided to the computer, for example, from a network or a storage medium. The storage medium may include one or more of a hard disk, a random access memory (RAM), a read-only memory (ROM), a storage device of a distributed computing system, an optical disc (a compact disc (CD), a digital versatile disc (DVD), a Blu-ray disc (BD), etc.), a flash memory device, a memory card, and the like.

The disclosure of the present exemplary embodiments includes the following configurations and method.

1. A cell detachment apparatus that detaches cells placed on a culture surface of a culture container for cells by applying vibration to the cells, the cell detachment apparatus comprising:
a vibrator and a vibration plate,
wherein the vibrator, the vibration plate, an acoustic transmission medium, and the culture container are arranged to be stacked on each other in this order, and the cell detachment apparatus is configured so that an area where the acoustic transmission medium comes into contact with the vibration plate does not overlap an area where the vibrator comes into contact with the vibration plate in a stacking direction of the vibrator, the vibration plate, an acoustic transmission medium, and the culture container.

2. The cell detachment apparatus according to configuration 1, wherein the vibration is vibration in an ultrasonic frequency range.

3. The cell detachment apparatus according to configuration 1 or 2, wherein the cell detachment apparatus is configured so that the acoustic transmission medium comes into contact with the vibration plate at least at one of antinode portions of a standing wave generated in the vibration plate by transmitting vibration of the vibrator to the vibration plate.

4. The cell detachment apparatus according to any one of configurations 1 to 3, wherein LN1/LM is less than 1.8, where LN1 is a length of the acoustic transmission medium in a direction perpendicular to the stacking direction, and LM is a distance from a first antinode portion of the standing wave generated in the vibration plate to a first node portion closest to the first antinode portion.

5. The cell detachment apparatus according to any one of configurations 1 to 3,
wherein a cross section of the acoustic transmission medium in a plane perpendicular to the stacking direction is circular, and
wherein LN2/LM is less than 1.8, where LN2 is a diameter of the cross section, and LM is a distance from a first antinode portion of the standing wave generated in the vibration plate to a first node portion closest to the first antinode portion.

6. The cell detachment apparatus according to any one of configurations 1 to 3, wherein the acoustic transmission medium is ring-shaped.

7. The cell detachment apparatus according to configuration 6, wherein LN3/LM is less than 1.8, where LN3 is a width of the ring-shaped acoustic transmission medium in a direction perpendicular to the stacking direction, and LM is a distance from a first antinode portion of the standing wave generated in the vibration plate to a first node portion closest to the first antinode portion.

8. The cell detachment apparatus according to any one of configurations 1 to 7, wherein the acoustic transmission medium includes a plurality of acoustic transmission media.

9. The cell detachment apparatus according to any one of configurations 1 to 8, wherein the acoustic transmission medium includes a polymer material.

10. The cell detachment apparatus according to configuration 9, wherein the polymer material includes silicone rubber.

11. The cell detachment apparatus according to any one of configurations 1 to 10, wherein a thermal conductivity of the acoustic transmission medium is 0.4 W/(m·K) or less.

12. The cell detachment apparatus according to any one of configurations 1 to 11, wherein a thickness of the acoustic transmission medium in the stacking direction is 0.1 mm or more and 3 mm or less.

13. The cell detachment apparatus according to any one of configurations 1 to 12, wherein a surface of the acoustic transmission medium on a side coming into contact with the culture container includes a convex curved surface.

14. The cell detachment apparatus according to any one of configurations 1 to 13, wherein the vibrator includes a ring-shaped piezoelectric material, a common electrode provided on a first surface of the piezoelectric material, and a driving electrode provided on a second surface of the piezoelectric material that is different from the first surface.

15. The cell detachment apparatus according to configuration 14, wherein a mechanical quality factor of the piezoelectric material is 1000 or more.

16. The cell detachment apparatus according to any one of configurations 1 to 15,
wherein the culture container has a protrusion portion on a surface opposite to the culture surface, and
wherein the cell detachment apparatus is configured so that the acoustic transmission medium and the vibration plate do not come into contact with the protrusion portion in a case where the vibrator, the vibration plate, the acoustic transmission medium, and the culture container are arranged to be stacked on each other in this order.

17. The cell detachment apparatus according to configuration 16, wherein the cell detachment apparatus is configured so that at least any one of the acoustic transmission medium and the vibration plate comes into contact with the protrusion portion via a cushioning material in the case where the vibrator, the vibration plate, the acoustic transmission medium, and the culture container are arranged to be stacked on each other in this order.

18. The cell detachment apparatus according to configuration 16 or 17, wherein a groove is provided in an area including a position where a position of the protrusion portion is projected to the vibration plate in the stacking direction.

19. The cell detachment apparatus according to any one of configurations 1 to 18, further comprising a control unit configured to apply vibration to the cells by driving the vibrator.

20. A method for detaching cells placed on a culture surface of a culture container for cells by applying vibration to the cells, the method comprising:
applying vibration to the cells using a cell detachment apparatus that includes a vibrator and a vibration plate, an acoustic transmission medium, and the culture container in a state where the vibrator, the vibration plate, the acoustic transmission medium, and the culture container are arranged to be stacked on each other in this order; and
applying vibration to the cells in a state where an area where the acoustic transmission medium comes into contact with the vibration plate does not overlap an area where the vibrator comes into contact with the vibration plate in the stacking direction.

The present disclosure is not limited to the above exemplary embodiments, and various modifications and variations can be made without departing from the spirit and scope of the present disclosure.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

1. A cell detachment apparatus that detaches cells placed on a culture surface of a culture container for cells by applying vibration to the cells, the cell detachment apparatus comprising:
a vibrator and a vibration plate,
wherein the vibrator, the vibration plate, an acoustic transmission medium, and the culture container are arranged to be stacked on each other in this order, and the cell detachment apparatus is configured so that an area where the acoustic transmission medium comes into contact with the vibration plate does not overlap an area where the vibrator comes into contact with the vibration plate in a stacking direction of the vibrator, the vibration plate, an acoustic transmission medium, and the culture container.

2. The cell detachment apparatus according to claim 1, wherein the vibration is vibration in an ultrasonic frequency range.

3. The cell detachment apparatus according to claim 1, wherein the cell detachment apparatus is configured so that the acoustic transmission medium comes into contact with the vibration plate at least at one of antinode portions of a standing wave generated in the vibration plate by transmitting vibration of the vibrator to the vibration plate.

4. The cell detachment apparatus according to claim 3, wherein LN1/LM is less than 1.8, where LN1 is a length of the acoustic transmission medium in a direction perpendicular to the stacking direction, and LM is a distance from a first antinode portion of the standing wave generated in the vibration plate to a first node portion closest to the first antinode portion.

5. The cell detachment apparatus according to claim 3,
wherein a cross section of the acoustic transmission medium in a plane perpendicular to the stacking direction is circular, and
wherein LN2/LM is less than 1.8, where LN2 is a diameter of the cross section, and LM is a distance from a first antinode portion of the standing wave generated in the vibration plate to a first node portion closest to the first antinode portion.

6. The cell detachment apparatus according to claim 3, wherein the acoustic transmission medium is ring-shaped.

7. The cell detachment apparatus according to claim 6, wherein LN3/LM is less than 1.8, where LN3 is a width of the ring-shaped acoustic transmission medium in a direction perpendicular to the stacking direction, and LM is a distance from a first antinode portion of the standing wave generated in the vibration plate to a first node portion closest to the first antinode portion.

8. The cell detachment apparatus according to claim 3, wherein the acoustic transmission medium includes a plurality of acoustic transmission media.

9. The cell detachment apparatus according to claim 1, wherein the acoustic transmission medium includes a polymer material.

10. The cell detachment apparatus according to claim 9, wherein the polymer material includes silicone rubber.

11. The cell detachment apparatus according to claim 1, wherein a thermal conductivity of the acoustic transmission medium is 0.4 W/(m·K) or less.

12. The cell detachment apparatus according to claim 1, wherein a thickness of the acoustic transmission medium in the stacking direction is 0.1 mm or more and 3 mm or less.

13. The cell detachment apparatus according to claim 1, wherein a surface of the acoustic transmission medium on a side coming into contact with the culture container includes a convex curved surface.

14. The cell detachment apparatus according to claim 1, wherein the vibrator includes a ring-shaped piezoelectric material, a common electrode provided on a first surface of the piezoelectric material, and a driving electrode provided on a second surface of the piezoelectric material that is different from the first surface.

15. The cell detachment apparatus according to claim 14, wherein a mechanical quality factor of the piezoelectric material is 1000 or more.

16. The cell detachment apparatus according to claim 1,
wherein the culture container has a protrusion portion on a surface opposite to the culture surface, and
wherein the cell detachment apparatus is configured so that the acoustic transmission medium and the vibration plate do not come into contact with the protrusion portion in a case where the vibrator, the vibration plate, the acoustic transmission medium, and the culture container are arranged to be stacked on each other in this order.

17. The cell detachment apparatus according to claim 16, wherein the cell detachment apparatus is configured so that at least any one of the acoustic transmission medium and the vibration plate comes into contact with the protrusion portion via a cushioning material in the case where the vibrator, the vibration plate, the acoustic transmission medium, and the culture container are arranged to be stacked on each other in this order.

18. The cell detachment apparatus according to claim 16, wherein a groove is provided in an area including a position where a position of the protrusion portion is projected to the vibration plate in the stacking direction.

19. The cell detachment apparatus according to claim 1, further comprising a control unit configured to apply vibration to the cells by driving the vibrator.

20. A method for detaching cells placed on a culture surface of a culture container for cells by applying vibration to the cells, the method comprising:
applying vibration to the cells using a cell detachment apparatus that includes a vibrator and a vibration plate, an acoustic transmission medium, and the culture container in a state where the vibrator, the vibration plate, the acoustic transmission medium, and the culture container are arranged to be stacked on each other in this order; and
applying vibration to the cells in a state where an area where the acoustic transmission medium comes into contact with the vibration plate does not overlap an area where the vibrator comes into contact with the vibration plate in the stacking direction.
